(19) United States
(12) Patent Application Publication
(10) Pub. No.: US 2025/0207894 A1
(43) Pub. Date: Jun. 26, 2025

(54) BODY SAFETY DEVICE WITH VISUAL SENSING AND HAPTIC RESPONSE USING ARTIFICIAL INTELLIGENCE

(71) Applicants: John Bridge, Hillsborough, NC (US); Frank L. Hammond, III, Atlanta, GA (US); Patrick Gibbons, Columbia, MD (US); Goktug Duman, Miami, FL (US); Arthi Abhyanker, Mountain View, CA (US); Joe Lanzi, Grover, MO (US); Nelson So, Dawsonville, GA (US); Raj Abhyanker, Mountain View, CA (US); Cameron Christensen, Mountain View, CA (US)

(72) Inventors: John Bridge, Hillsborough, NC (US); Frank L. Hammond, III, Atlanta, GA (US); Patrick Gibbons, Columbia, MD (US); Goktug Duman, Miami, FL (US); Arthi Abhyanker, Mountain View, CA (US); Joe Lanzi, Grover, MO (US); Nelson So, Dawsonville, GA (US); Raj Abhyanker, Mountain View, CA (US); Cameron Christensen, Mountain View, CA (US)

(21) Appl. No.: 18/596,684

(22) Filed: Mar. 6, 2024

Related U.S. Application Data
(60) Provisional application No. 63/614,022, filed on Dec. 22, 2023.
(60) Provisional application No. 63/616,817, filed on Jan. 1, 2024.
(60) Provisional application No. 63/622,514, filed on Jan. 18, 2024.
(60) Provisional application No. 63/626,075, filed on Jan. 29, 2024.
(60) Provisional application No. 63/552,265, filed on Feb. 12, 2024.
(60) Provisional application No. 63/555,014, filed on Feb. 17, 2024.
(60) Provisional application No. 63/554,360, filed on Feb. 16, 2024.

(51) Int. Cl. F41H 1/00; A61B 5/00; G06F 40/58; G08B 6/00; G08B 21/02

(52) U.S. Cl. CPC F41H 1/00; A61B 5/6805; A61B 5/747; G06F 40/58; G08B 6/00; G08B 21/02

(57) ABSTRACT

Disclosed are a method, system, and apparatus of s body safety device with visual sensing and haptic response using artificial intelligence. In one embodiment, a body worn safety device includes a visual sensor integrated into a tactical gear and a haptic feedback device integrated in the tactical gear. The haptic feedback device vibrates when the visual sensor detects an ambient threat to a wearer of the tactical gear. The haptic feedback device may detect the ambient threat using an artificial intelligence based threat detection model. The visual sensor may be part of an array of visual sensors around a torso of the wearer such that ambient threats around the wearer are captured through the artificial intelligence based threat detection model. A battery unit may be detachable from the tactical gear.

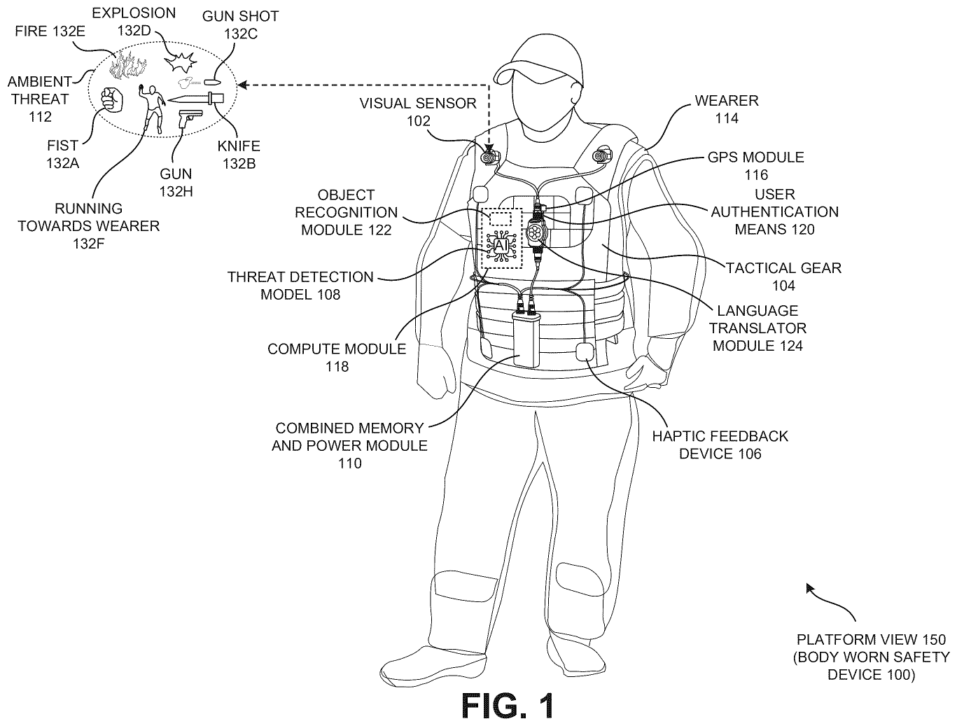

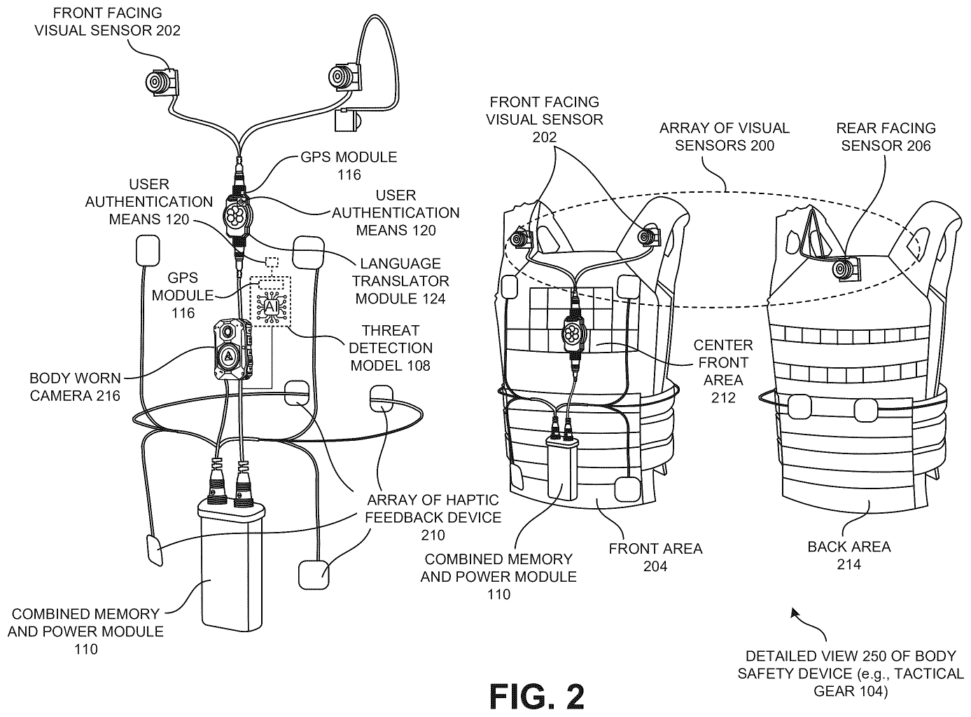

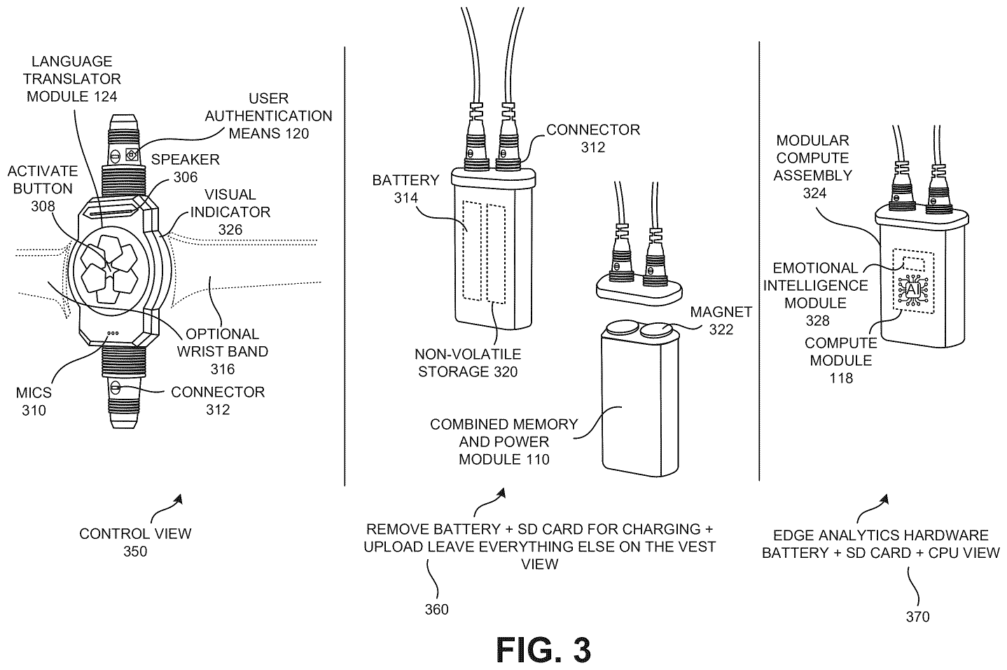

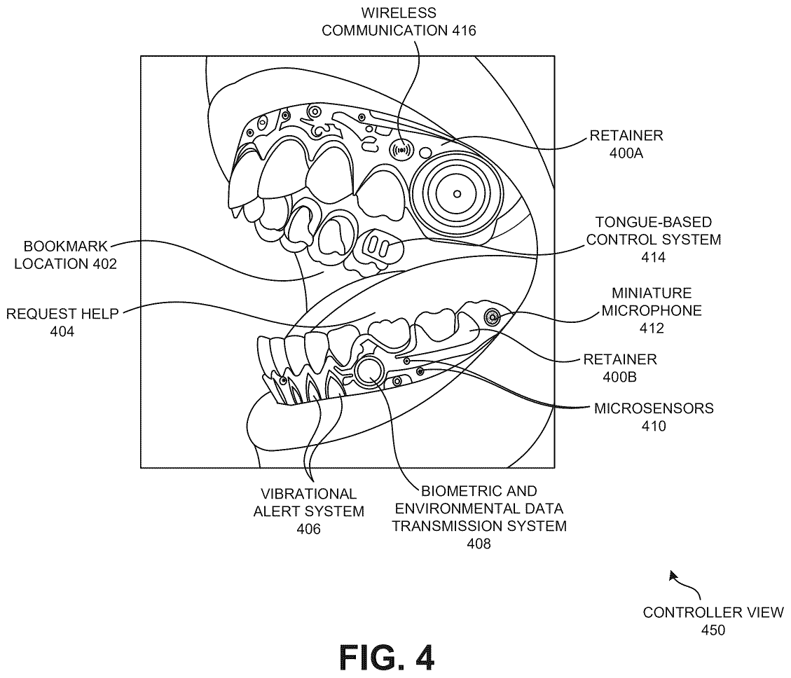

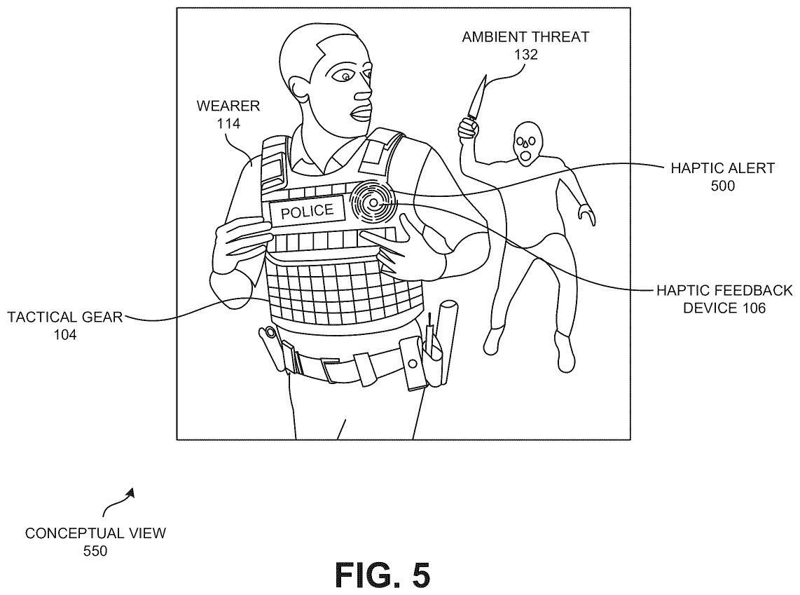

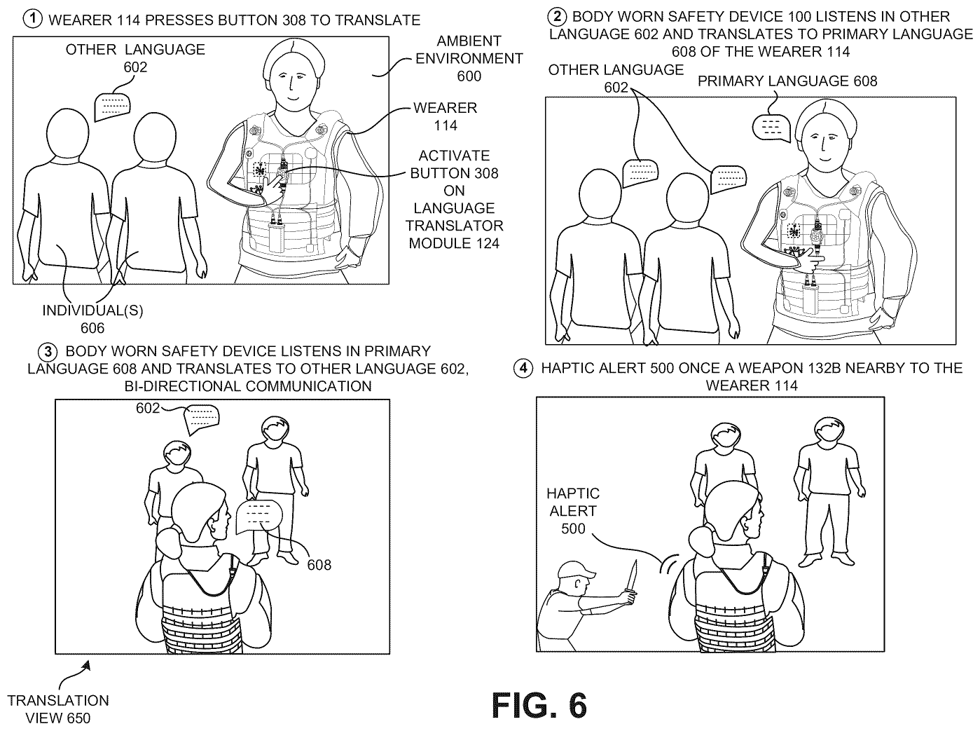

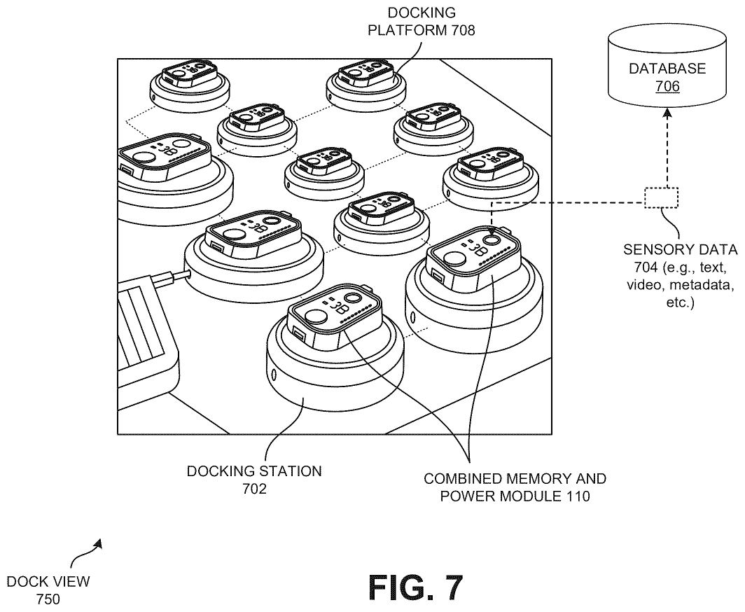

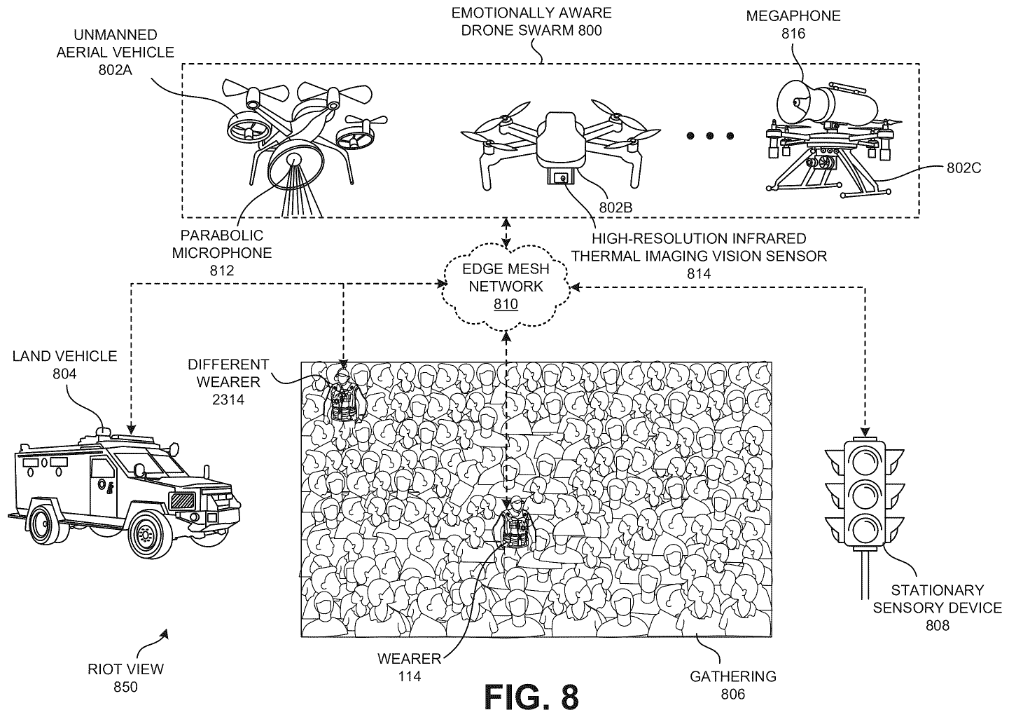

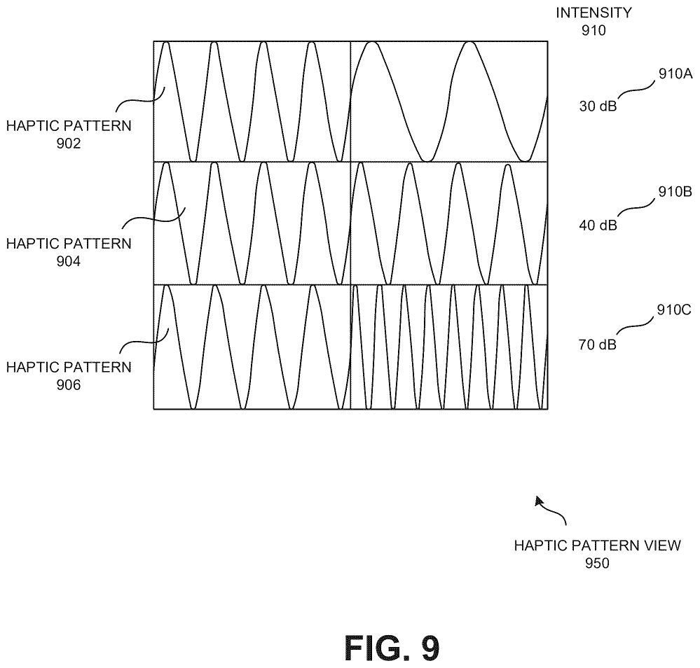

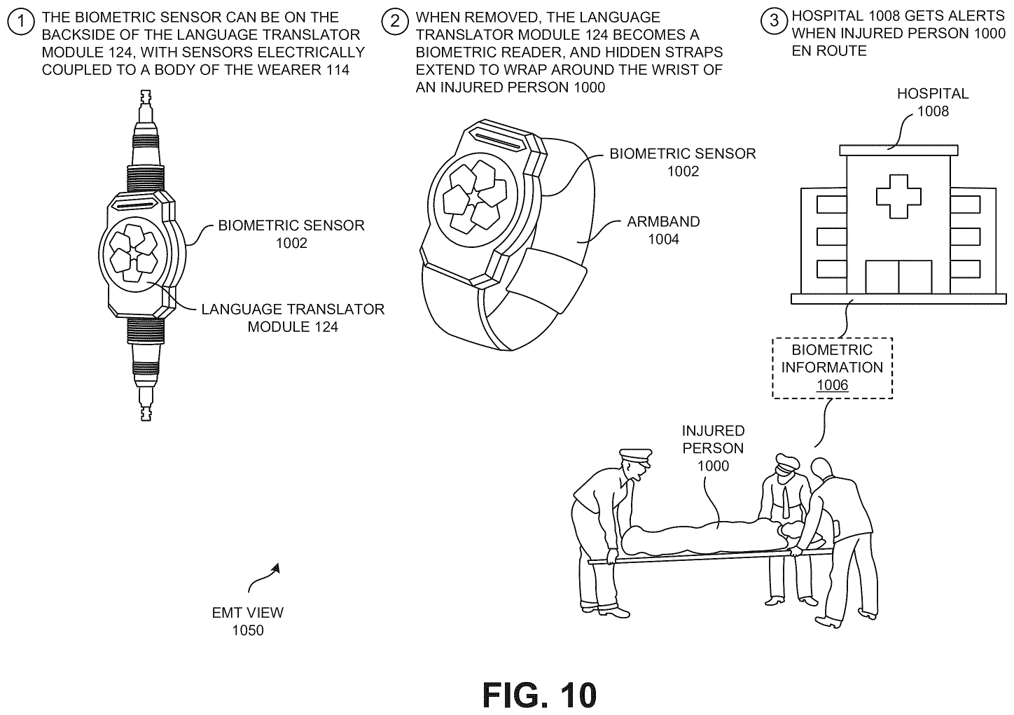

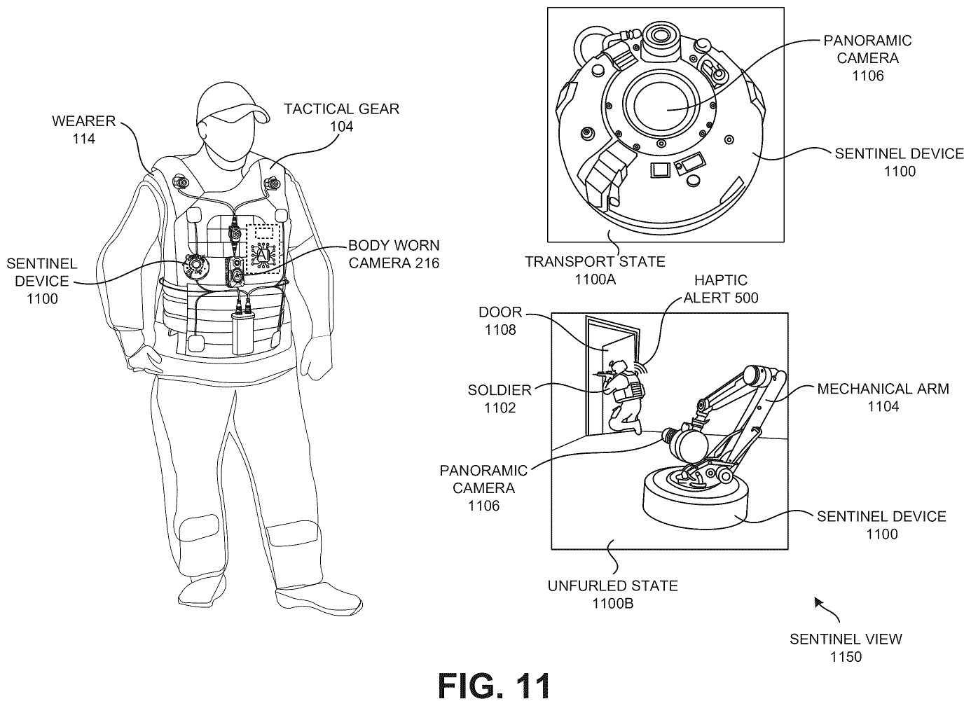

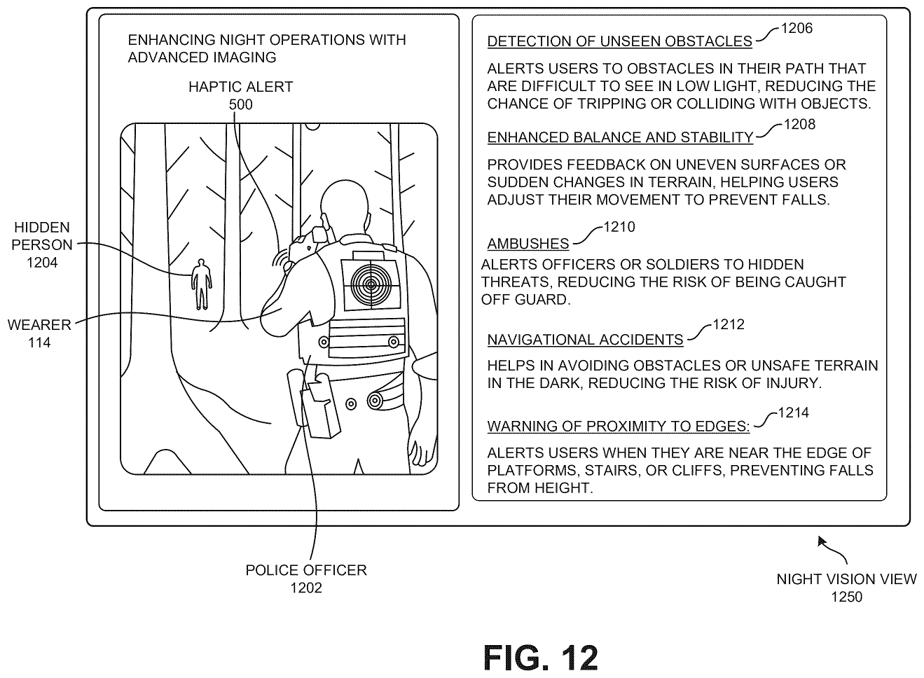

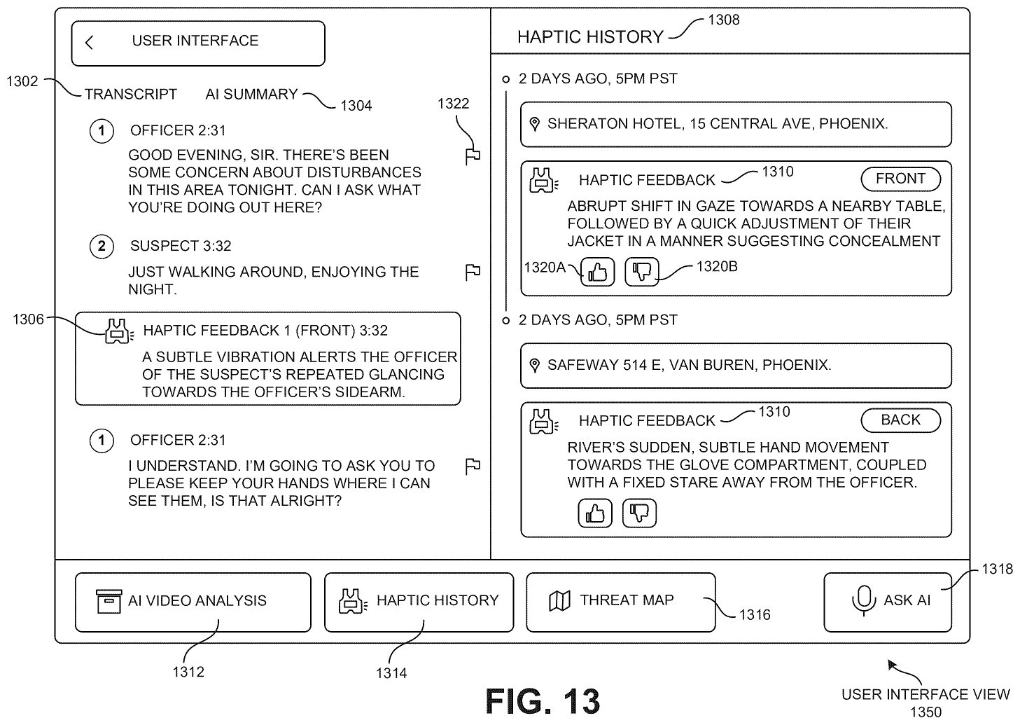

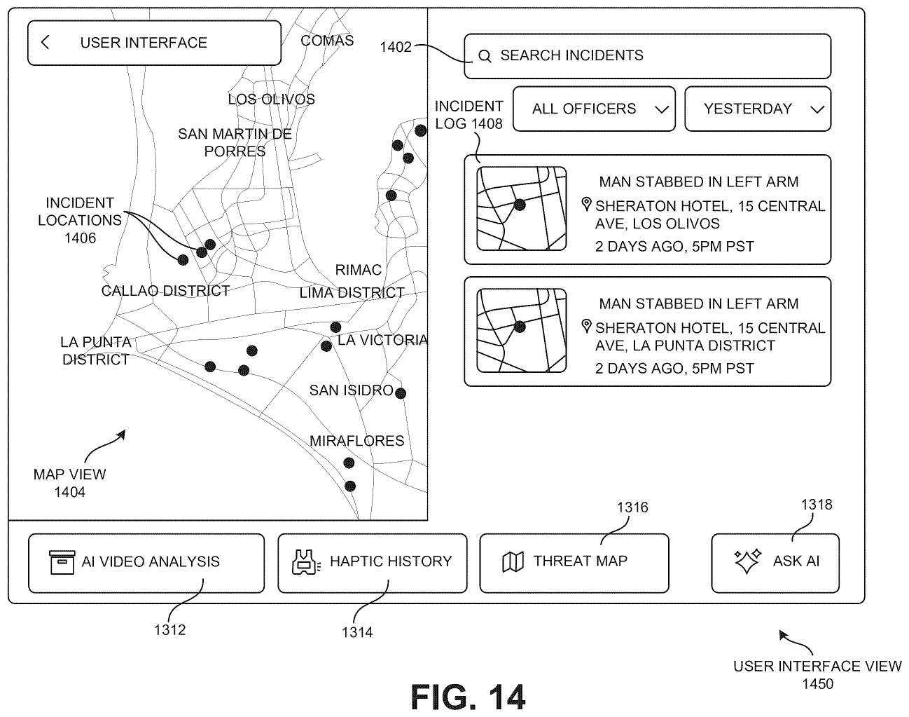

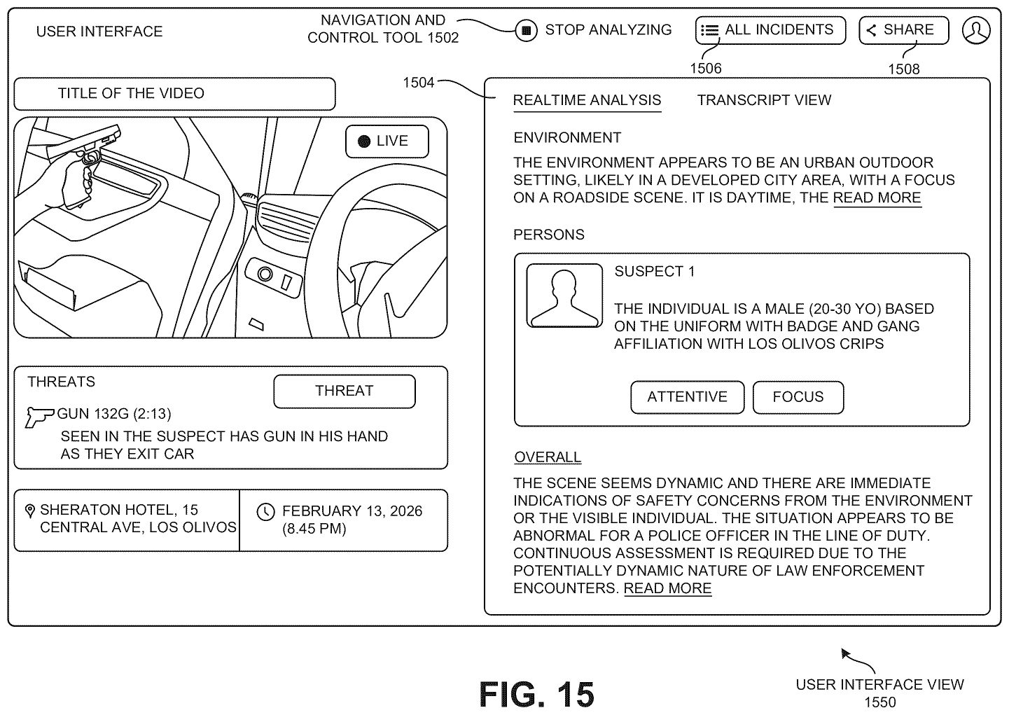

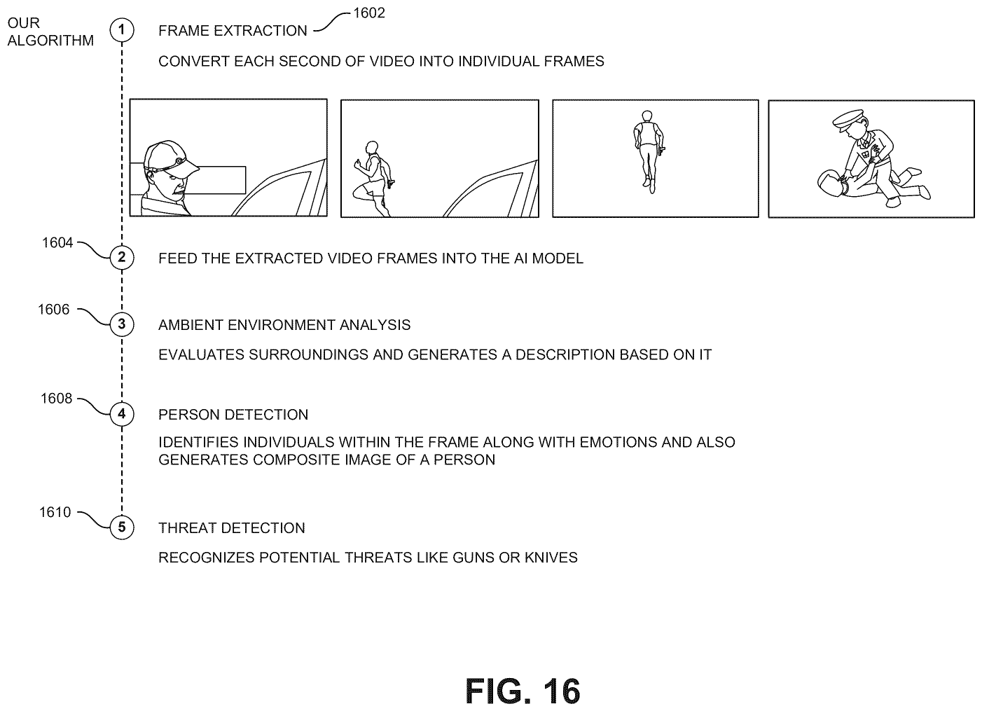

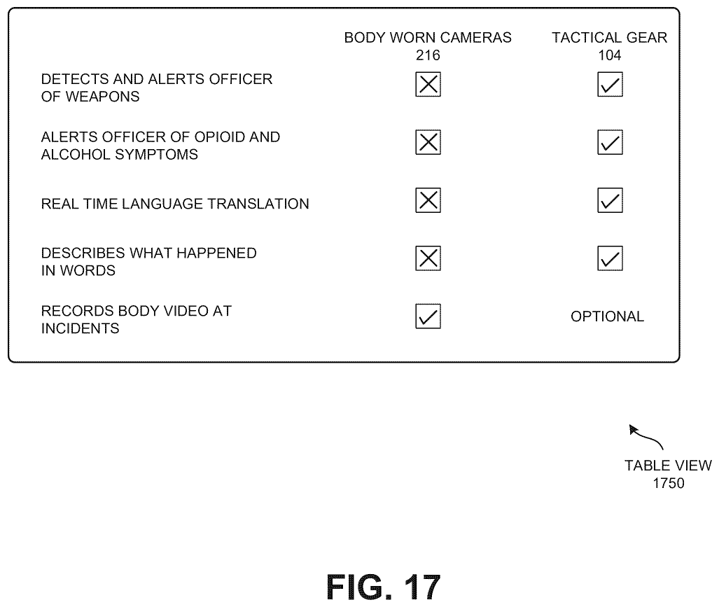

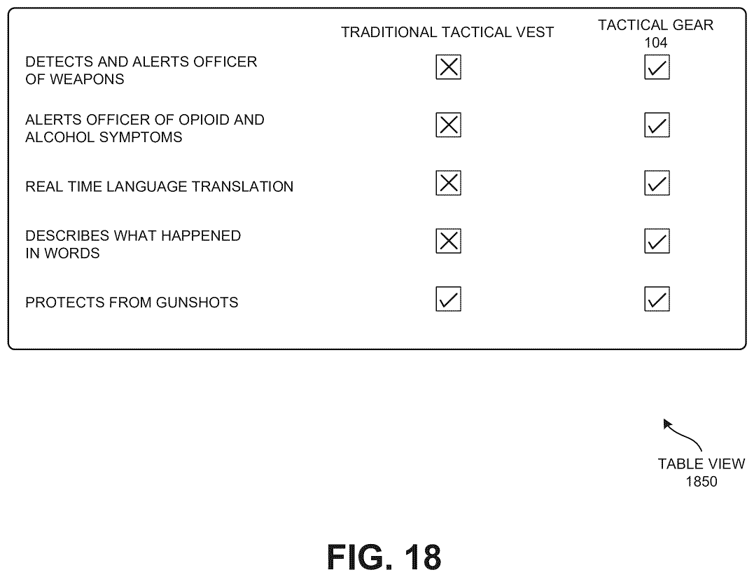

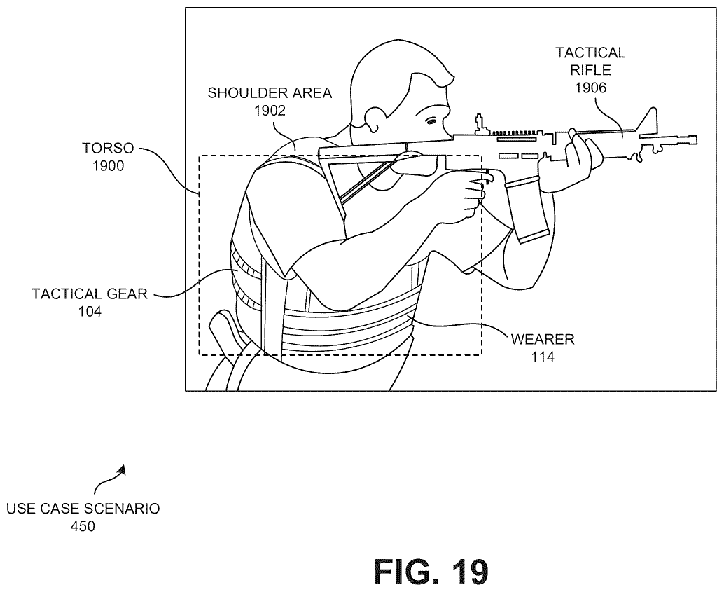

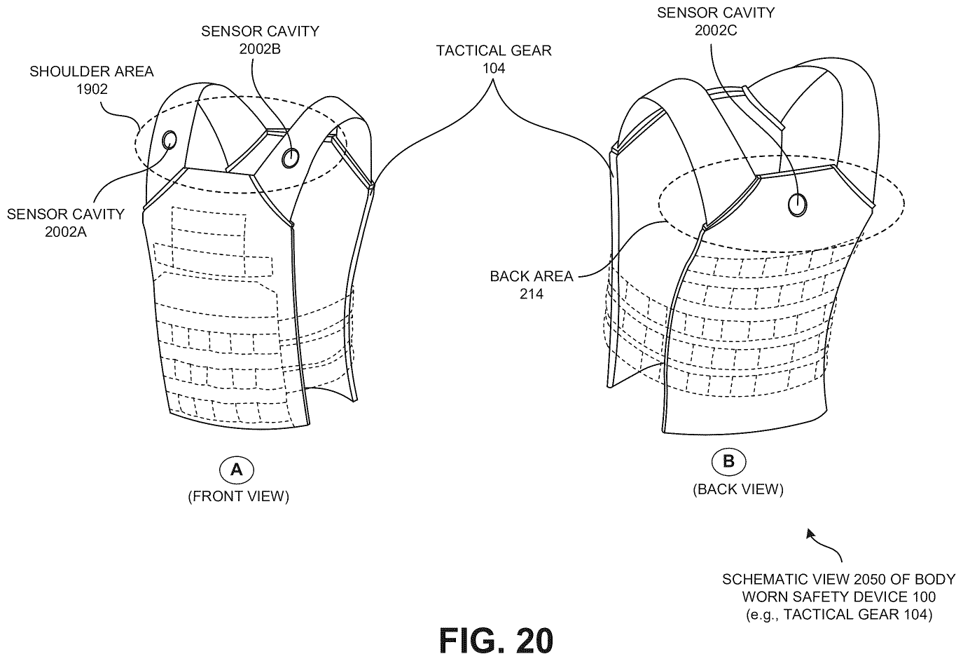

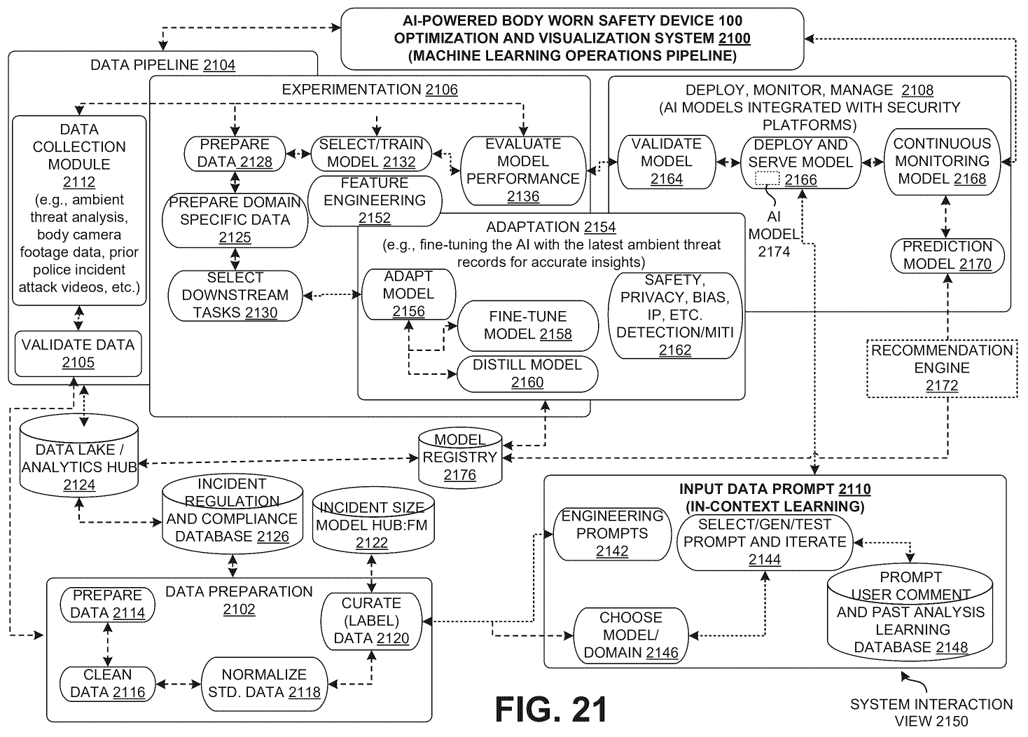

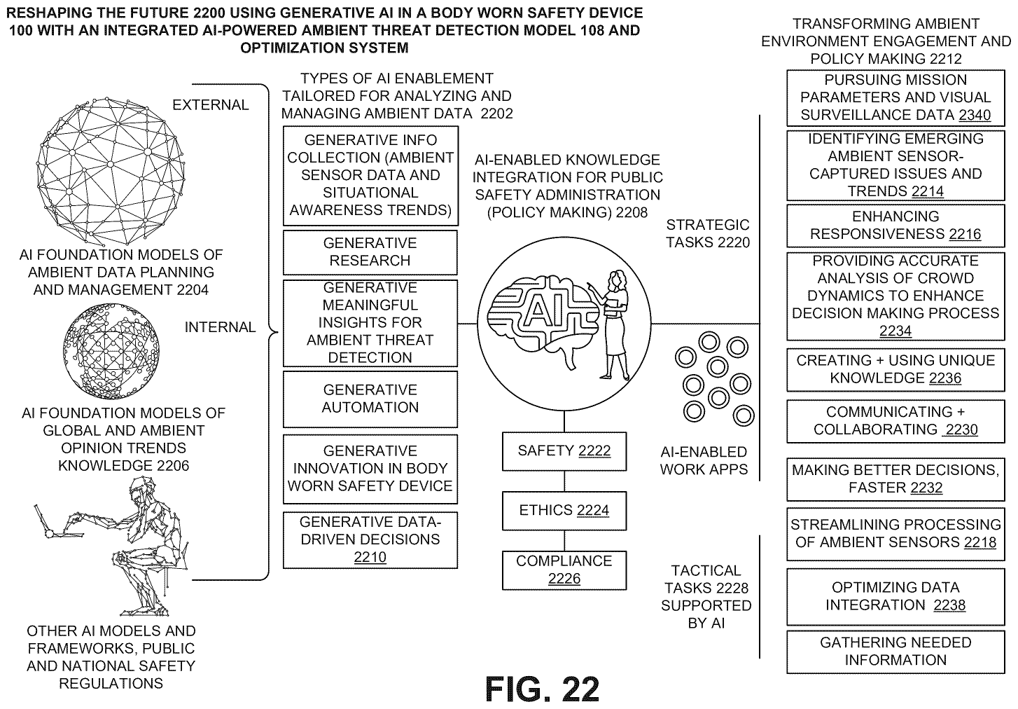

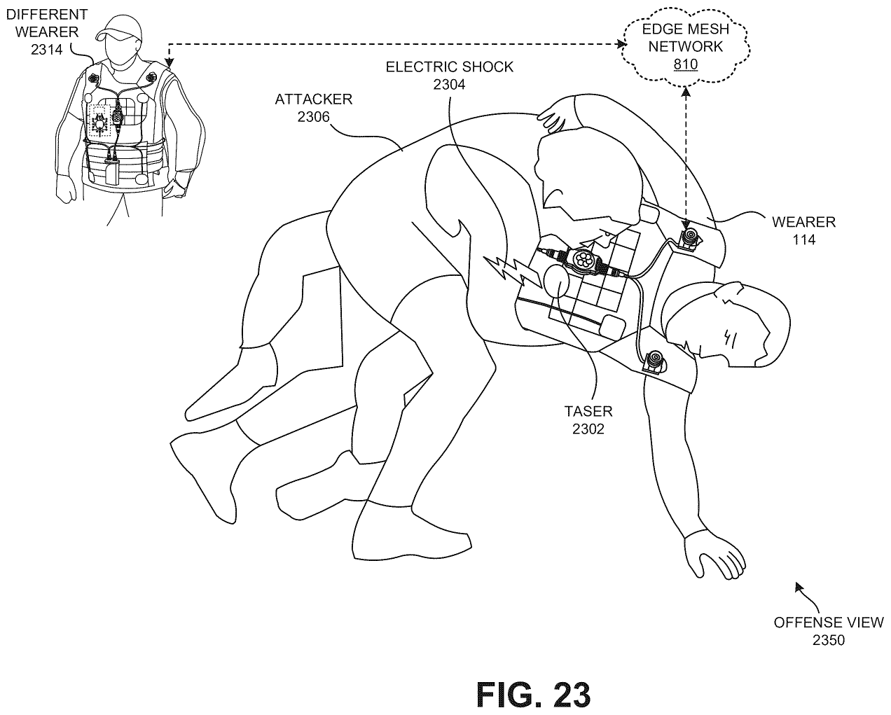

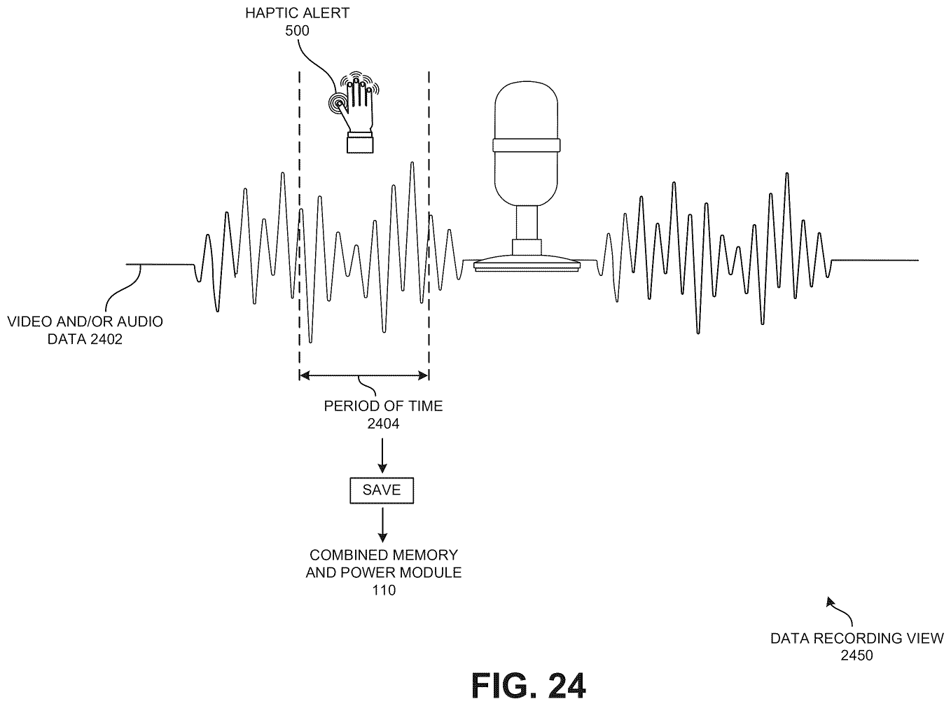

### CLAIM OF PRIORITY

This Application is a conversion Application of, claims priority to, and incorporates by reference herein the entirety of the disclosures of:
U.S. Provisional Patent Application No. 63/614,022 titled MULTI-FUNCTIONAL WEARABLE AI-ENABLED PENDANT APPARATUS, SYSTEM, AND METHOD OF AMBIENT DATA ANALYSIS AND COMMUNICATION IN LAW ENFORCEMENT, FIRE, MEDICAL RESPONDER, PRIVATE SECURITY, JOURNALISM, COMMERCIAL AND MILITARY OPERATIONAL ENVIRONMENTS filed on Dec. 22, 2023,
U.S. Provisional Patent Application No. 63/616,817 titled EMOTIONALLY INTELLIGENT AERIAL DRONE SYSTEM FOR ENHANCED SITUATIONAL AWARENESS AND RESPONSIVE OPERATIONS filed on Jan. 1, 2024,
U.S. Provisional Patent Application No. 63/622,514 titled HAPTIC FEEDBACK RESPONSIVE TO A THREAT IDENTIFIED THROUGH A GENERATIVE ARTIFICIAL INTELLIGENCE BODY WORN APPARATUS filed on Jan. 18, 2024,
U.S. Provisional Patent Application No. 63/626,075 titled SECURE EDGE MESH NETWORK SYSTEM FOR ENHANCED VISUAL INTERPRETATION AND REAL-TIME SITUATIONAL AWARENESS IN COMBAT ZONES filed on Jan. 29, 2024,
U.S. Provisional Patent Application No. 63/552,265 titled MODULAR INTEGRATED BODY CAMERA SYSTEM FOR ENHANCED ERGONOMICS, OPERATIONAL EFFICIENCY, AND TECHNOLOGICAL ADAPTABILITY IN LAW ENFORCEMENT EQUIPMENT filed on Feb. 12, 2024, and
U.S. Provisional Patent Application No. 63/554,360 titled ENHANCED SITUATIONAL AWARENESS THROUGH A HAPTIC WEARABLE DEVICE OF A POLICE OFFICER OR A WARFIGHTER, ACTIVATED BY A NEARBY NETWORKED VEHICLE OR A STATIONARY SENSOR UPON DETECTING A THREAT filed on Feb. 16, 2024; and
U.S. Provisional Patent Application No. 63/555,014 titled TRAUMATIC INJURY COMMUNICATION METHODOLOGY AND SYSTEM THROUGH A BODY WORN DEVICE filed on Feb. 17, 2024.

### FIELD OF TECHNOLOGY

The present disclosure relates generally to the field of wearable personal safety technology. This disclosure relates generally to a body safety device with visual sensing and haptic response using artificial intelligence.

### BACKGROUND

Police and military peacekeepers may face threats that they may not immediately perceive. These threats can conic in various forms and may not always be easily identifiable. For example, in conflict zones and volatile areas, peacekeepers may not always be aware of hidden dangers like landmines or improvised explosive devices (IEDs). These threats can be concealed and may not be immediately visible, posing a significant risk to the safety of peacekeepers. Hostile forces may plan and execute ambushes against peacekeeping forces. These ambushes can occur suddenly, catching peacekeepers off guard and leading to casualties. In some situations, combatants may blend in with the civilian population, making it difficult for peacekeepers to identify potential threats. This can create a situation where peacekeepers do not perceive the danger until it's too late. Snipers and sharpshooters can pose a threat from a distance, making it challenging for peacekeepers to perceive their presence until shots are fired.

Insurgent groups may employ guerrilla tactics, hit-and-run attacks, and hit-and-hide strategies. These tactics can create an environment where peacekeepers may not always perceive the imminent threat. Enemy combatants might infiltrate peacekeeping camps or units, posing as friendly forces. This infiltration can lead to internal threats that peacekeepers may not initially perceive. Psychological warfare can be used to manipulate perceptions and create confusion among peacekeepers. Disinformation and propaganda can make it difficult to accurately assess threats.

In active combat zones such as in the Middle East or Ukraine, there are various potential threats that military personnel may encounter. Warfighters and police may be faced with ambushes and surprise attacks, as insurgents may exploit the terrain and urban environments for cover and concealment. This can be particularly acute in low light and nighttime conditions, where potholes, hidden persons, and/or navigational challenges can pose difficulties. In areas with tall buildings or dense urban areas, snipers and sharpshooters can pose a significant threat to military personnel. IEDs placed along patrol routes are a constant danger, with insurgents frequently using them to target military convoys. There may be a risk of attacks using non-conventional methods, such as chemical or biological agents, which can have devastating effects.

Insurgent groups may employ guerrilla warfare tactics, including blending in with the civilian population and launching hit-and-run attacks. Unexploded bombs or mines from previous conflicts that remain active and dangerous can be a hazard in combat zones. The use of small unmanned aerial vehicles (UAVs) equipped with cameras or explosives for surveillance or direct attacks is a growing concern. Electronic interference, such as jamming or intercepting communications and disrupting electronic devices, can hinder military operations. In addition, individuals carrying explosive devices on their person, intending to detonate them in close proximity to military targets, may pose a significant and immediate threat.

In densely populated urban areas, individuals may carry concealed firearms or weapons. Police officers may not always perceive the presence of these weapons until a situation escalates, posing a significant danger. Criminal activities such as drug dealing, illegal gambling, or human trafficking can occur discreetly within urban neighborhoods. Officers may need to rely on intelligence and investigative work to uncover these hidden threats, which may not always be possible. Gangs operating within urban environments can be a constant threat. Gang members may blend into the community, making it difficult for officers to identify them until a confrontation arises.

Urban areas may witness protests and civil unrest. These events can quickly escalate, with officers facing threats like projectiles, Molotov cocktails, and/or aggressive crowds may not be immediately anticipated. In densely populated urban areas, hostage situations can develop rapidly, and officers may not perceive a threat until it is too late. Pursuits through city streets can endanger officers and the public. The threat of a suspect fleeing at high speeds may not be immediately apparent until a pursuit begins. Criminals may plan ambushes against police officers, especially during routine patrols or traffic stops. These ambushes can catch officers off guard. Responding to domestic violence calls can be unpredictable. Officers may not always perceive the potential for violence until they arrive at the scene, making de-escalation skills crucial. Officers can sometimes encounter individuals in mental health crises. These situations can quickly escalate, with officers unable to assess the level of threat and respond accordingly.

### SUMMARY

Disclosed are a method, system, and apparatus of a body safety device with visual sensing and haptic response using artificial intelligence.

In one aspect, a body worn safety device includes a visual sensor integrated into a tactical gear and a haptic feedback device is integrated in the tactical gear. The haptic feedback device vibrates when the visual sensor detects an ambient threat to a wearer of the tactical gear.

The haptic feedback device may detect the ambient threat using an artificial intelligence based threat detection model. The visual sensor may be part of an array of visual sensors around the torso of the wearer. The artificial intelligence based threat detection model may detect and/or analyze human emotion of an individual in an ambient area to the wearer using computer vision and/or auditory sensing to detect the presence and/or absence of the ambient threat. The visual sensor may be a front-facing visual sensor embedded in both shoulder areas of the tactical gear such that the wearer is able to sense the ambient threat through the haptic feedback device when the ambient threat approaches the wearer of the tactical gear. The visual sensor may be embedded in a center back area of the tactical gear such that the wearer may be able to sense the ambient threat through the haptic feedback device when the ambient threat approaches from behind the wearer of the tactical gear.

A language translator module may be integrated in a center front area through which the wearer is able to bi-directionally communicate with an individual in the ambient environment using any language other than a primary language spoken by the wearer when the language translator module is activated.

A combined memory and power module may be removable from the tactical gear. The combined memory and power module may power the visual sensor and/or the haptic feedback device. The combined memory and power module may include a memory storage which auto downloads sensory data captured from the visual sensor and the haptic feedback device when the combined memory and power module is docked in a docking station. The combined memory and power module may simultaneously charge multiple devices on the wearer including a mobile phone, a body worn camera, and/or the visual sensor. A processing unit may be removable from the tactical gear and which provides optional processing and sensor capabilities to the body worn safety device. A user authentication means may activate the visual sensor and the haptic feedback device if the wearer is an authorized user.

The visual sensor may serve as a visual recording device. The haptic feedback device may be part of an array of haptic feedback device to vibrate at different locations of a body of the wearer depending on a directional location of the ambient threat. An intensity of vibration of the haptic feedback device may be dependent on the proximity of the ambient threat to the wearer. A pattern of vibration of the haptic feedback device may be dependent on a type of threat. The visual sensor may automatically document a health condition of an injured individual in the ambient environment and communicate that information to a nearby hospital where the injured individual is to be transported. The haptic feedback device may vibrate when a different wearer of a different body worn safety device detects a different ambient threat in the same ambient environment to the wearer. The different body worn safety device may be communicatively coupled with the body worn safety device through an ad-hoc edge mesh network formed between the body worn safety device and the different body worn safety device.

The body worn safety device may solely be a real time observation device that provides early warnings to the wearer of any one of a pre-assaultive threat, an opioid addiction warning, an intoxicated state warning, and a mental health warning in an ambient environment.

The body worn safety device may vibrate when an unmanned aerial vehicle networked with the body worn safety device in an active incident area perceives an ambient threat nearby to the wearer. The unmanned aerial vehicle may be part of an emotionally aware drone swarm which uses any one of a plurality of sensors to detect aggressive emotions in a riotous gathering. The body worn safety device may vibrate when a land vehicle networked with the body worn safety device perceives an ambient threat nearby to the wearer. Only a subset of an array of haptic feedback device on the body worn safety device closest to the ambient threat may vibrate based on a directionality of the ambient threat. The body worn safety device may include a global positioning system.

The body worn safety device may also vibrate when a stationary sensory device networked with the body worn safety device perceives an ambient threat nearby to the wearer. The visual sensor may be embedded in a flush manner in a shoulder area of the tactical gear such that movement of a tactical rifle is not impeded by the visual sensor. The body worn safety device may not permanently store real-time threat analysis.

A biometric sensor of the tactical gear may measure the biometric information of the wearer. The biometric sensor may be detachable from the tactical gear and placeable on an injured person nearby to the wearer through an armband extendable from the biometric sensor when it is removed from the tactical gear. The biometric sensor may automatically store and/or communicate the biometric information of the injured person to a hospital when removed from the tactical gear and placed on an arm of the injured person, when the injured person is en route to the hospital.

A single-chip Software-Defined Vision Sensor (SDVS) with integrated neuromorphic sensing and processing may deliver real time vision intelligence with privacy at source to the body worn safety device to detect an obstacle and/or the ambient threat in a path of the wearer during low light conditions and trigger the haptic feedback device when the obstacle and/or the ambient threat is detected.

A spherical disc may be wirelessly coupled with the body worn safety device that is carryable and/or throwable by the wearer. The spherical disc may automatically unfurl when it is thrown by the wearer onto a level surface, such that a camera of the spherical disc is perpendicularly manifested and actively records when in the unfurled state. The haptic feedback device on the body worn safety device may vibrate when an ambient threat is visible to the camera of the spherical disc in the unfurled state.

A retainer placed in a mouth of the wearer may bookmark a geospatial location based on an action in the mouth of the wearer and/or to request help to arrive at the geospatial location. The body worn safety device may communicate without electronic signature through a non-radiative, human-body-centered communication network. The body worn safety device may include a taser integrated into the tactical gear to automatically deliver an electric shock to an attacker who is physically wrestling with the wearer when the artificial intelligence based threat detection model detects a physical assault. The visual sensor may be a thermal imaging sensor that detects heat signatures emitted by objects and converts them into visual data. An audiovisual data of the visual sensor closest to the haptic feedback device may be automatically stored in a combined memory and power module for a configurable one of (1) a short period of time prior to the vibration of the haptic feedback device, (2) during the vibration of the haptic feedback device, and/or (3) another short period of time after the vibration of the haptic feedback device stops vibrating.

In another aspect, a body worn safety device, includes an array of visual sensors around a torso of a wearer such that ambient threats around the wearer are captured through an artificial intelligence based threat detection model, and an array of haptic feedback device to vibrate at different locations of a body of the wearer depending on a directional location of the ambient threat using the artificial intelligence based threat detection model.

In yet another aspect, a body worn safety device, includes a visual sensor embedded in a center front area of the tactical gear such that the wearer is able to sense the ambient threat through the haptic feedback device when the ambient threat approaches from the front of the wearer of the tactical gear. A language translator module is integrated with center front area through which the wearer is able to bi-directionally communicate with an individual in the ambient environment using any language other than a primary language spoken by the wearer when the language translator module is activated and a haptic feedback device integrated in the tactical gear vibrates when the visual sensor detects an ambient threat to a wearer of the tactical gear.

The methods and systems disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and the detailed description that follows.

1. A body worn safety device, comprising:
a visual sensor integrated into a tactical gear; and
a haptic feedback device integrated in the tactical gear,
wherein the haptic feedback device to vibrate when the visual sensor detects an ambient threat to a wearer of the tactical gear, and at least one of:
an intensity of vibration of the haptic feedback device is dependent on a proximity of the ambient threat to the wearer, and
a pattern of vibration of the haptic feedback device is dependent on a type of threat.

2. The body worn safety device of claim 1 wherein the body worn safety device to detect the ambient threat using an artificial intelligence based threat detection model.

3. The body worn safety device of claim 2:
wherein the visual sensor is part of an array of visual sensors around a torso of the wearer, and
wherein the artificial intelligence based threat detection model to detect and analyze human emotion of an individual in an ambient area to the wearer using computer vision and auditory sensing to detect the presence or absence of the ambient threat.

4. The body worn safety device of claim 1 wherein the visual sensor is a front-facing visual sensor embedded in both shoulder areas of the tactical gear such that the wearer is able to sense the ambient threat through the haptic feedback device-when the ambient threat approaches the wearer of the tactical gear.

5. The body worn safety device of claim 1 wherein the visual sensor is embedded in a center back area of the tactical gear such that the wearer is able to sense the ambient threat through the haptic feedback device when the ambient threat approaches from behind the wearer of the tactical gear.

6. The body worn safety device of claim 1 wherein a language translator module is integrated in a center front area through which the wearer is able to bi-directionally communicate with an individual in the ambient environment using any language other than a primary language spoken by the wearer when the language translator module is activated.

7. The body worn safety device of claim 1 further comprising:
a combined memory and power module which is removable from the tactical gear and which provides power to the visual sensor and the haptic feedback device,
wherein the combined memory and power module includes a memory storage which auto downloads sensory data captured from the visual sensor and the haptic feedback device when the combined memory and power module is docked in a docking station, and
wherein in the combined memory and power module to simultaneously charge multiple devices on the wearer comprising any one of a mobile phone, a body worn camera, and the visual sensor.

8. The body worn safety device of claim 1 further comprising a processing unit which is removable from the tactical gear and which provides optional processing and sensor capabilities to the body worn safety device.

9. The body worn safety device of claim 1 further comprising a user authentication means to activate the visual sensor and the haptic feedback device the wearer is an authorized user, and wherein the visual sensor also serves as a visual recording device.

10. The body worn safety device of claim 1 wherein the haptic feedback device is part of an array of haptic feedback devices to vibrate at different locations of a body of the wearer depending on a directional location of the ambient threat.

11. (canceled)

12. The body worn safety device of claim 1 wherein the visual sensor to automatically document a health condition of an injured individual in the ambient environment and communicate that information to a nearby hospital where the injured individual is to be transported.

13. The body worn safety device of claim 1:
wherein the haptic feedback device to vibrate when a different wearer of a different body worn safety device to detect a different ambient threat in a same ambient environment of the wearer, and
wherein the different body worn safety device is communicatively coupled with the body worn safety device through an ad-hoc edge mesh network formed between the body worn safety device and the different body worn safety device.

14. The body worn safety device of claim 1 wherein the body worn safety device is solely a real time observation device that provides early warnings to the wearer of any one of a pre-assaultive threat, an opioid addiction warning, an intoxicated state warning, and a mental health warning in an ambient environment.

15. The body worn safety device of claim 1:
wherein the body worn safety device to vibrate when an unmanned aerial vehicle networked with the body worn safety device in an active incident area perceives an ambient threat nearby to the wearer, and
wherein the unmanned aerial vehicle is part of an emotionally aware drone swarm which uses any one of a plurality of sensors to detect aggressive emotions in a riotous gathering.

16. The body worn safety device of claim 1 wherein the body worn safety device to vibrate when a land vehicle networked with the body worn safety device perceives an ambient threat nearby to the wearer.

17. The body worn safety device of claim 1 wherein only a subset of an array of haptic feedback devices on the body worn safety device closest to the ambient threat to vibrate based on a directionality of the ambient threat, and wherein the body worn safety device to include a global positioning system.

18. The body worn safety device of claim 1 wherein the body worn safety device to vibrate when a stationary sensory device networked with the body worn safety device perceives an ambient threat nearby to the wearer.

19. The body worn safety device of claim 1 wherein the visual sensor is embedded in a flush manner in a shoulder area of the tactical gear such that movement of a tactical rifle is not impeded by the visual sensor.

20. The body worn safety device of claim 1 wherein the body worn safety device does not permanently store real-time threat analysis.

21. The body worn safety device of claim 1 further comprising a biometric sensor of the tactical gear to measure a biometric information of the wearer,
wherein the biometric sensor is detachable from the tactical gear and placeable on an injured person nearby to the wearer through an armband extendable from biometric sensor when it is removed from the tactical gear, and
wherein the biometric sensor to automatically communicate the biometric information of the injured person to a hospital when removed from the tactical gear and placed on an arm of the injured person, when the injured person is en route to the hospital.

22. The body worn safety device of claim 1 wherein the body worn safety device to detect at least one of an obstacle and the ambient threat in a path of the wearer during low light conditions and to trigger the haptic feedback device when the at least one of the obstacle and the ambient threat is detected.

23. The body worn safety device of claim 1 further comprising a spherical disc wirelessly coupled with the body worn safety device that is carryable by the wearer and is throwable by the wearer, wherein the spherical disc to automatically unfurl when it is thrown by the wearer onto a level surface, such that a camera of the spherical disc is perpendicularly manifested and actively recording when in the unfurled state, and wherein the haptic feedback device on the body worn safety device to vibrate when ambient threat is visible to the camera of the spherical disc in the unfurled state.

24. The body worn safety device of claim 1 wherein a retainer placed in a mouth of the wearer to at least one of bookmark a geospatial location based on an action in the mouth of wearer, and request help to arrive at the geospatial location.

25. The body worn safety device of claim 1 wherein the body worn safety device communicates without electronic signature through a non-radiative, human-body-centered communication network.

26. The body worn safety device of claim 1 further comprising a taser integrated into the tactical gear to automatically deliver an electric shock to an attacker who is physically wrestling with the wearer when the artificial intelligence based threat detection model detects a physical assault.

27. The body worn safety device of claim 1 wherein an audiovisual data of the visual sensor closest to the haptic feedback device is automatically stored in a combined memory and power module for at least one of a short period of time prior to the vibration of the haptic feedback device, during the vibration of the haptic feedback device, and another short period of time after the vibration of the haptic feedback device stops vibrating.

28. The body worn safety device of claim 1 wherein the visual sensor is a thermal imaging sensor that detects heat signatures emitted by objects and converts them into visual data.

29. A body worn safety device, comprising:
a visual sensor integrated into a tactical gear; and
a haptic feedback device integrated in the tactical gear,
wherein the haptic feedback device to vibrate when the visual sensor detects an ambient threat to a wearer of the tactical gear, and
wherein the body worn safety device to detect at least one of an obstacle and the ambient threat in a path of the wearer during low light conditions and to trigger the haptic feedback device when the at least one of the obstacle and the ambient threat is detected.

30. A body worn safety device, comprising:
a visual sensor integrated into a tactical gear; and
a haptic feedback device integrated in the tactical gear,
wherein the haptic feedback device to vibrate when the visual sensor detects an ambient threat to a wearer of the tactical gear, and
wherein the body worn safety device is a real time observation device that provides early warnings to the wearer of any one of a pre-assaultive threat, an opioid addiction warning, an intoxicated state warning, and a mental health warning in an ambient environment.

31. A body worn safety device, comprising:
a visual sensor integrated into a tactical gear; and
a haptic feedback device integrated in the tactical gear,
wherein the haptic feedback device to vibrate when the visual sensor detects an ambient threat to a wearer of the tactical gear, and at least one of:
an intensity of vibration of the haptic feedback device is dependent on a proximity of the ambient threat to the wearer, and
a pattern of vibration of the haptic feedback device is dependent on a type of threat, and
wherein the body worn safety device to vibrate when an unmanned aerial vehicle networked with the body worn safety device in an active incident area perceives an ambient threat nearby to the wearer.
